(19) United States
(12) Patent Application Publication
(10) Pub. No.: US 2009/0176265 A1
(43) Pub. Date: Jul. 9, 2009

(54) METHOD OF ASSESSING BIOLOGICAL TEST SPECIMEN

(76) Inventors: John M. Stencel, Lexington, KY (US); Haiping Song, Lexington, KY (US); Clair L. Hicks, Nicholasville, KY (US); Frederick A. Payne, Lexington, KY (US)

Correspondence Address: KING & SCHICKLI, PLLC, 247 NORTH BROADWAY, LEXINGTON, KY, 40507, US

(21) Appl. No.: 12/278,599

(22) PCT Filed: Feb. 7, 2007
(86) PCT No.: PCT/US07/03312

§ 371 Date: Nov. 7, 2008

Related U.S. Application Data
(60) Provisional application No. 60/765,926, filed on Feb. 7, 2006.

(51) Int. Cl. C12Q 1/02

(52) U.S. Cl. 435/29

(57) ABSTRACT

A method of assessing a biological test specimen includes the steps of detecting an acoustic emission produced by the biological test specimen and comparing the detected acoustic emission to a compilation of acoustic emissions produced by known biological sources. The method further includes the monitoring of various attributes of the test specimen.

This application claims the benefit of U.S. Provisional Patent Application Ser. No. 60/765,926 filed on 7 Feb. 2006.

### TECHNICAL FIELD

The present invention relates generally to the field of biology and, more particularly, to the dynamic assessment of cell and micro types and their interactions with cells and viruses.

### BACKGROUND OF THE INVENTION

The cell is the basic unit of life. Based on the organization of their cellular structures, all living cells can be divided into two groups: prokaryotic and eukaryotic. Animals, plants, fungi, protozoans, and algae all possess eukaryotic cell types. Only bacteria have prokaryotic cell types.

Bacteria play important roles in human, animal and plant life, global ecosystems, industry and medicine. Some types of bacteria sustain and nurture life, some are critical for industrial processes, and some cause debilitating disease and death. All bacteria have one characteristic: they lack a cell nucleus. They are the most numerous organisms on earth; consist of only a single cell; have cell membranes; grow in a wide variety of habitats and conditions; are generally quite small (˜micrometer dimensions) and have quite simple shapes.

The detection and identification of bacteria are, typically, difficult and time consuming. Real-time measurement of cell and microbe types and concentrations, the presence and activity of bacteriophages (viruses) in colonies of bacteria, and the detection of viruses and viral infections require specialty detectors and peripheral equipment. No broadly applicable, real-time cell and microbe monitoring instrumentation is currently commercially available. The present invention has been conceived for the purpose of providing real-time assessment of microbes and cells, their processes and activities during their growth, stationary and declining phases. The present invention employs the detection and analysis of characteristic or ‘fingerprint’ acoustic emission (AE) from microbes and cells to provide dynamic assessments of their presence and activity, and to identify cell and microbe types and their interactions with cells and viruses.

### SUMMARY OF THE INVENTION

In accordance with the purposes of the present invention as described herein, a method is provided for assessing a biological test specimen. That method comprises detecting an acoustic emission (AE) produced by the biological test specimen and comparing the detected AE to a compilation of acoustic emissions (AEs) produced by known biological sources. The method may also include the step of providing an impulse to the biological test specimen to produce a detectable response. That impulse may be selected from a group of impulses including an electrical impulse, a chemical impulse, a magnetic impulse, an acoustic impulse, a vibratory impulse, an electromagnetic impulse, a mechanical impulse, a radioactive impulse and any combinations thereof. In addition, the method may include the detecting of the acoustic emission in a range of between about 20 to about 5,000,000 Hertz.

The method includes using an acoustic sensor to detect the AE. That sensor may be selected from a group of sensors consisting of a piezoelectric sensor, a capacitive sensor, an optoelectronic sensor, a magnetorestrictive sensor and combinations thereof. Multiple sensors may be used to detect the AE. Alternatively, a single sensor may be used to detect the AE by moving the sensor across the biological test specimen.

The present method allows the monitoring of a number of different attributes of the test specimen by detecting and analyzing the AE. Thus, the method may include the steps of monitoring:

a. nutrients/chemical and bi-product imbibing and expulsion;

b. cell growth and cell division;

c. cell movement;

d. plasma ejection and imbibing;

e. natural cell wall vibration;

f. cell expiration;

g. quorum sensing communication;

h. transfer of genetic material via sex pili;

i. growth of pili and transport of biofilm material through the pili;

j. Brownian motion;

k. flagella rotation.

In the following description there is shown and described several preferred embodiments of this invention, simply by way of illustration of some of the modes best suited to carry out the invention. As it will be realized, the invention is capable of other different embodiments and its several details are capable of modification in various aspects all without departing from the invention. Accordingly, the drawings and descriptions will be regarded as illustrative in nature and not as restrictive.

### BRIEF DESCRIPTION OF THE DRAWINGS

The accompanying drawings incorporated in and forming a part of this specification, illustrate several aspects of the present invention, and together with the description serve to explain certain principles of the invention. In the drawings:

FIG. 1 is a diagram of an AE waveform presented to illustrate the relationship of the waveform to chemical and physical actions of bacteria;

FIG. 2 is a diagram illustrating a typical bacterial growth and death curve;

FIG. 3 is a diagram illustrating a logarithmic cell and microbe growth rate;

FIG. 4 is a diagram illustrating a logarithmic cell and microbe death rate;

FIG. 5 is a graph illustrating the number of AE hits-vs-time for E. coli 15CC, 15DD and 15Q mixed colony acquired using a hydrophone and contact sensor;

FIG. 6 is a graph illustrating the logarithmic values of the number of hits during the rapid increase in AE signal of FIG. 5;

FIG. 7 is a graph illustrating the logarithmic values of the number of hits during the decline in AE signal of FIG. 5;

FIG. 8 is a graph comparing the AE number of hits-vs-time from three different E. coli strains using an AE contact sensor with an effective frequency range of approximately 5 to approximately 50 kHz;

FIG. 9 is a graph comparing the AE number of hits-vs-time from two different E. coli strains using an AE contact sensor with an effective frequency range of approximately 50 to approximately 200 kHz.

FIG. 10 is a graph illustrating AE and CFU data for E. coli 15Q.CFU(x 5) data was multiplied by 5 to place the cfu curve near and to compare it to the acoustic data accept for square data point at 8.5 hours. Vertical axis is relative value ie value divided by an average from beginning of test; and

FIG. 11 is a graph comparing the CFU and AE data for E. coli 15CC.

### DETAILED DESCRIPTION OF THE PREFERRED EMBODIMENTS OF THE INVENTION

Acoustic emission or AE is defined as “the class of phenomena whereby transient elastic waves are generated by a rapid release of energy from localized sources within a material or structure or the transient waves so generated”. The localized sources are the bacteria and other organisms associated with and/or interacting with the bacteria. A typical AE range to be used is between 0.000020-to-1 MHz (i.e. 20 Hz to 5,000,000 Hz) although even higher AE frequencies can be used (ie up to 10,000,000 Hz).

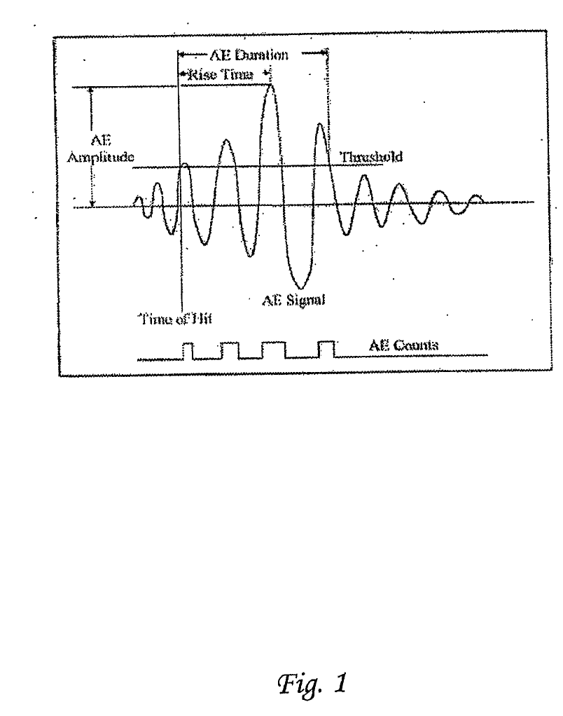

A diagram of an AE waveform is presented in FIG. 1 to better understand its relationship to chemical and physical actions of bacteria. The diagram depicts the amplitude of an acoustic signal with time. These minute energy pulses are associated with compression of longitudinal waves, have durations that typically are between microseconds-to-milliseconds and are caused by physical and/or chemical impulses generated during chemical/physical processes and motions.

The detection of AE can be accomplished using commercially available sensors, for example acoustic sensors using piezoelectric, capacitive, fiber Bragg grating (optoelectronic) or magnetorestrictive components. Such sensors are available from various manufacturers including but not limited to Physical Acoustics, Inc, Siemens, and Festo AG & Co. Amplitude-versus-time and amplitude-versus-frequency are extractable from each signal/waveform. Because of the fast response times available (˜μseconds), the pulse rise times and amplitudes, and pulse durations and their energies are also measurable even for pulses having durations between microseconds-to-milliseconds. The amplitude-time information in FIG. 1 is defined as “time-domain data”; “frequency-domain data” are established by fast Fourier transform (FFT) mathematical manipulations of the “time domain data”.

The assessment and identification of prokaryotic cells, eukaryotic cells and viruses, and processes in which they are involved, are accomplished by examining AE data, and then creating ‘fingerprints’ of their activity. For example, computer-based logic software which distinguishes ‘fingerprints’ of cells and associated chemical and/or physical processes occurring within, on, between or to them and their environment is used to ‘teach’ a computer what feedback is required for achieving a desired product or operation effected by cell activity, and for identifying the type(s) of cell or microbe cell present. Simultaneously, by knowing the intensity of the AE it is possible to measure dynamically and in real-time cell and microbe concentrations. Knowledge of the ‘fingerprint’ and/or concentration then enables the use of electrical, electromagnetic, magnetic and/or mechanical feedback or change in growth conditions (temperature, substrate concentration, pH, etc) to thereby control processes in which they are involved. The present invention provides a method to sense, monitor and control processes in which cells and microbes are present and/or used.

The monitoring of cell and microbe activity via AE is a ‘passive’ mode of acoustic sensing, i.e. it entails ‘listening’ to the cells and characterizing the emitted signal. It is also possible to provide an impulse to the cells, whether electrical, chemical, magnetic, acoustic, vibration, electromagnetic, mechanical, radioactive, etc., and then to ‘listen’ to and understand the cell and microbe response.

The principal behind AE sensors for monitoring and identifying cells is that the root mean square level of the AE is proportional to the amount of acoustic energy radiated during cell and microbe ‘activities’. The closeness of some AE sensors to such activities, for example, piezoelectric detectors, determines the intensity of the AE signal because the sound pressure level (SPL) produced is inversely proportional to distance, 1/d, between the emission source and the detector. Hence, acoustic intensities detected from nearby sources would be greater than from far away sources. Nevertheless, multiple acoustic sensors or transversal of a region using a single sensor still would provide characteristic AE from cell and microbe sources.

Many industrial processes using cell and microbe cultures—e.g., fermentation or the production of cottage cheese—are batch-wise processes in which prescribed concentrations and types of bacteria are added after establishing needed reactant conditions. After the cultures are added, the bacteria grow in number during which time they consume a substrate or reactants and produce the desired products. As the reactant substrate is consumed and/or the products of growth accumulate, the cell and microbe growth rate declines, reaches a steady state. When the substrate is exhausted or products of growth reach toxic levels the bacteria population then begins to decline.

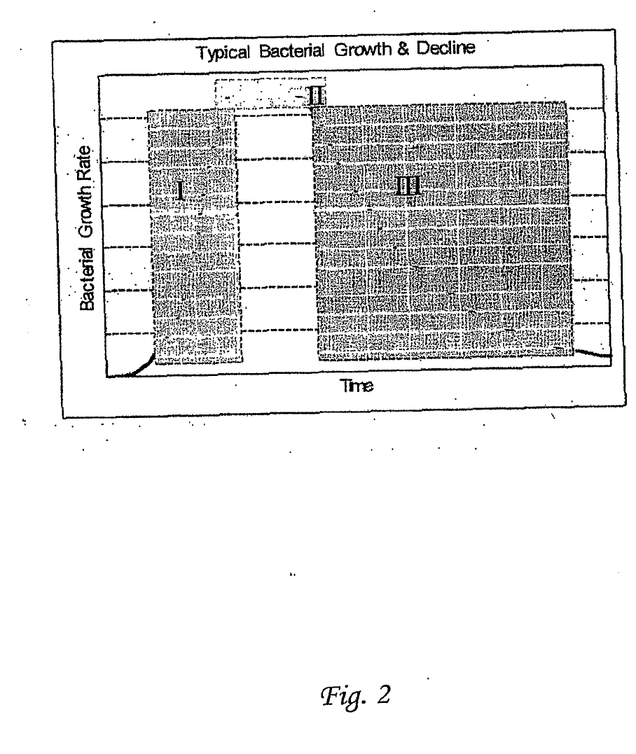

A typical bacterial growth and death curve is displayed in FIG. 2. Region I represents rapid growth in the number of bacteria (defined as the log phase); Region II represents a stationary phase, caused by negative interactions such as competition for food; Region III is a decline or death phase.

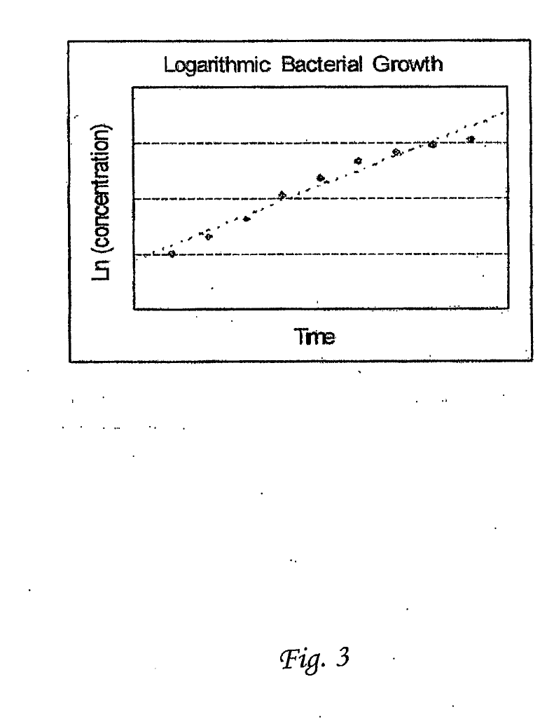

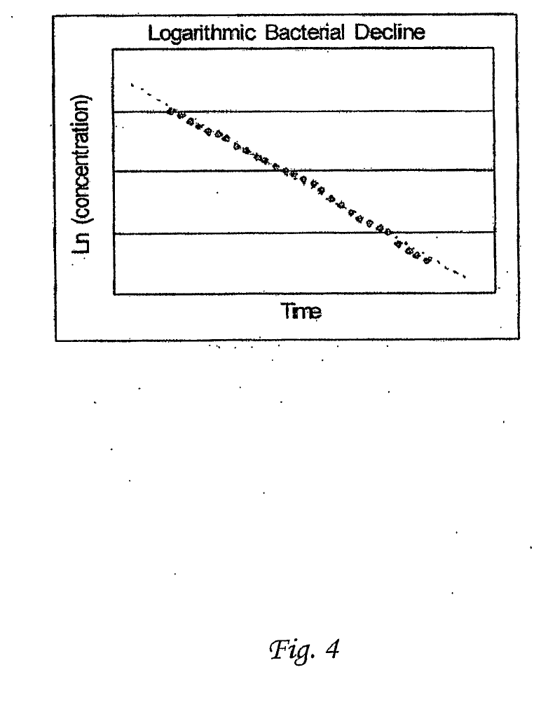

Before rapid growth phase of Region I, there is a ‘lag phase’ where cells are acclimating to the new environment after inoculation and are preparing their metabolic system for growth. Even so, the cells are actively metabolizing, in preparation for cell division. The rate of cell division and death has exponential dependency, i.e. mathematical examination of Region I and Region III are both represented by exponential functions. The natural logarithm of the values within these regions present linear dependencies, either increasing or decreasing linearly with time (see FIGS. 3 and 4).

Cell activities during these three phases are multifaceted, and include:
a. nutrient/chemical and byproduct imbibing and expulsion;
b. cell growth (from metabolizing nutrients and before division);
c. cell division by simple binary fission;
d. cell movement (‘taxis’) toward or away from chemical and/or physical stimuli;
e. flagella rotation;
f. growth of pili and the transport of biofilm materials through them;
g. transfer of genetic material via sex pili;
h. plasmid ejection and imbibing;
i. quorum sensing communication;
j. cell expiration;
k. cell wall vibration associated with movement; and
l. natural cell vibration.

Each of these activities is considered to have the potential to produce measurable AE signals, categorized into a few distinguishing groups:

A) The transport of nutrients, chemicals, molecules, plasmids, byproducts, genetic and biofilm materials through pili and the cytoplasmic membrane (lipid bilayer and protein structures) involve transport and diffusion via/through minute tubular structures and holes. It is known that the transport of air through minute holes produces AE in the 200-300 KHz region.

The expulsion of gaseous byproducts or cell metabolism may also produce microbubbles within the medium in which the bacteria reside. The generation of bubbles relates to cell and microbe activity; bubble generation, breakage and coalescence are also known to produce AE.

B) Cell growth and division involve the replication of the single DNA molecule within the cell, attachment of the two DNA to different parts of the cell membrane and then the cell elongation and division, whereby the replicate and original chromosomes are separated. Upon pulling apart, the cell splits (cytokinesis) producing two cells of identical genetic composition. This process is expected to produce AE during the various steps associated with chemical transport, and cell wall distortion, disruption and growth.

C) Cellular wall motions/undulations, flagella rotation and ‘wagging’, pili attachment and growth involve movements of the cell wall and physical structures within and on the cell. Rotating flagella (with some at frequencies between 200-to-1000 Hz) and the concerted motion creates net translational movement; ‘wagging’ flagella also creates a net translational velocity. Pili growth via the addition of a single protein at the end of the pili and then coordinated bacteria ‘rides’ on the pili cause step-wise motion; attachment of pili to a surface/substrate and then their shortening enables cells to be pulled toward a location or the site of the pili attachment. The movement of bacteria that do not have flagella may also involve surface waves on and within the cell wall (3). Each of these processes that move bacteria would produce weak AE signals.

Cell and microbe taxis causes translational movement up to an equivalent of 50 cell diameters per second, although cell movement speeds are more likely near micrometers-per-second range. This speed is slow and would create or have low frequency AE signals. However, step-wise and/or surface wave motions are rapid, thereby creating high frequency AE signals.

D) Quorum-sensing communication between bacteria involves the release of small molecules that float among the bacteria and deliver chemical messages via docking with another cell. When bacteria determine via quorum-sensing communication that a critical mass of organisms have assembled, they may move, slide, or glide toward each other and construct a thick, slimy biofilm for community protection. In some cases, the molecular release is in the form of minute vesicles (˜50 nm in diameter). These vesicles consist of a lipid bilayer surrounding an aqueous core which can transport lipid-soluble toxins on their surface and protein toxins in their core. The vesicles can release their poisonous cargo by fusing with the lipid bilayer of target cells. Such release is coordinated with huge numbers of cells performing identical activities and involves diffusion through the cytoplasm via tubular structures and holes. Similar to (A) above, high frequency AE is anticipated.

E) Cell expiration can be caused by the lack of nutrients and exposure to environments, other cells and microphages or viruses. When phages puncture or erode holes through cell and microbe membranes, massive, transitory leakage of ions may occur. If infected with phage DNA or RNA, the DNA or RNA will replicate within the bacteria and within minutes the host cell will lyse causing the eruption of progeny phages. These occurrences can create AE signals associated with molecular diffusion and cell rupture. For the latter, and because cell and microbe cells can be conceived as ‘bubbles’, the AE of rupture is expected to be related to the natural frequency of the ‘bubble’, i.e. inversely related to cell radius. Because cell and microbe cells are micrometers in size, the bursting AE frequencies would be in 1/(10⁻⁶)˜10⁶ Hz, I.E. NEAR 1,000,000 Hz.

F) Bacteria not having flagella may move by Brownian (molecular) motion. The vibration rate of the Brownian motion is inversely proportional to the size of the cell. For example, rapid Brownian movement is a common characteristic of non-motile cocci such as Staphylococcus, Streptococcus, Lactococcus, or Micrococcus. An inverse proportionality is also common to the natural vibrational frequency (ω) of volumetric oscillations of flexible films, given by ω=[√(3γP/ρ][1/r], where r is the cell and microbe radius, ρ is its density, P is the pressure and γ is the ratio of specific heats. Thus, “fingerprint” oscillations may be generated due to cell and microbe size and membrane densities.

Preliminary Data

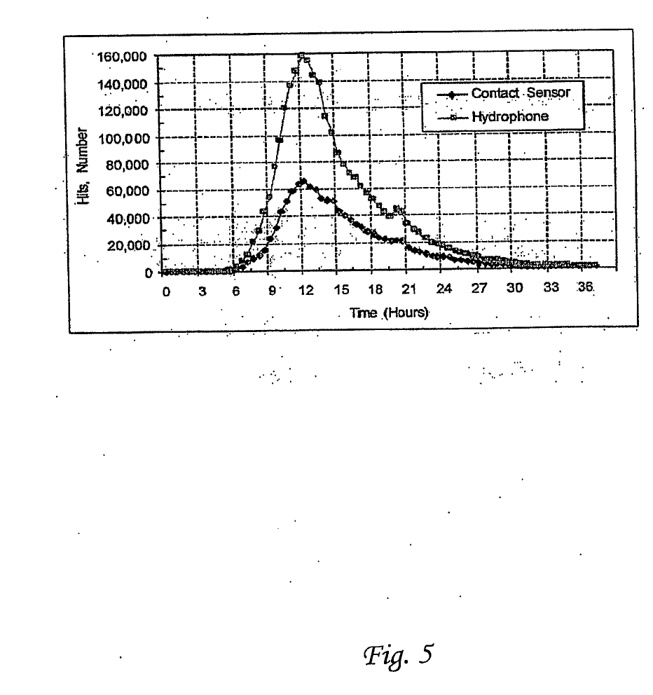

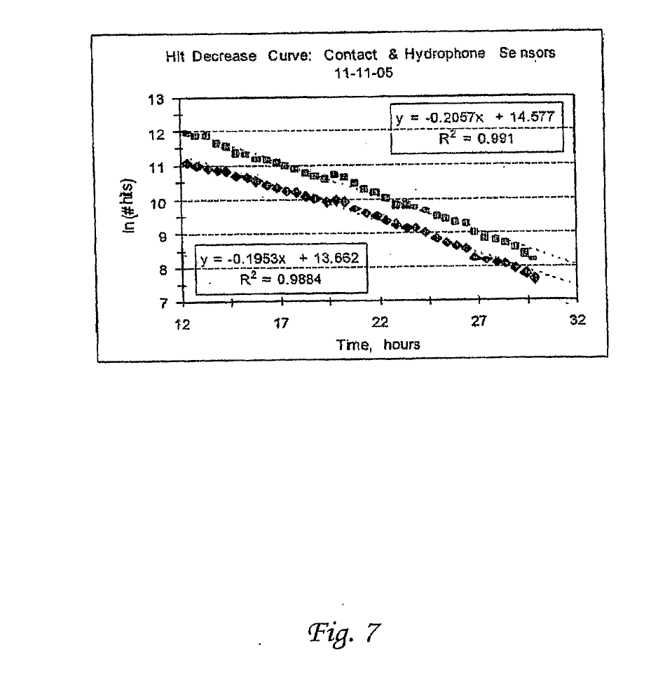

Data acquired using the present method are displayed in FIGS. 5-11. FIG. 5 shows the number of AE hits-versus-time acquired using contact and hydrophone sensors mounted onto and within a growth chamber containing an inoculated growth medium during the growth, stationary, and death phases for three-strains of bacteria, including Escherichia coli 15cc, 15dd and 15q. Broth was inoculated with 2% culture at Time=0 hours. After six hours the number of hits from AE signals began to increase rapidly until 12 hours was attained, after which the number of hits began to decrease. During this decline in the number of hits, three data plateaus were observed by both hydrophone and contact sensors at Times=14 hours; =20.5 hours; =24.5 hours.

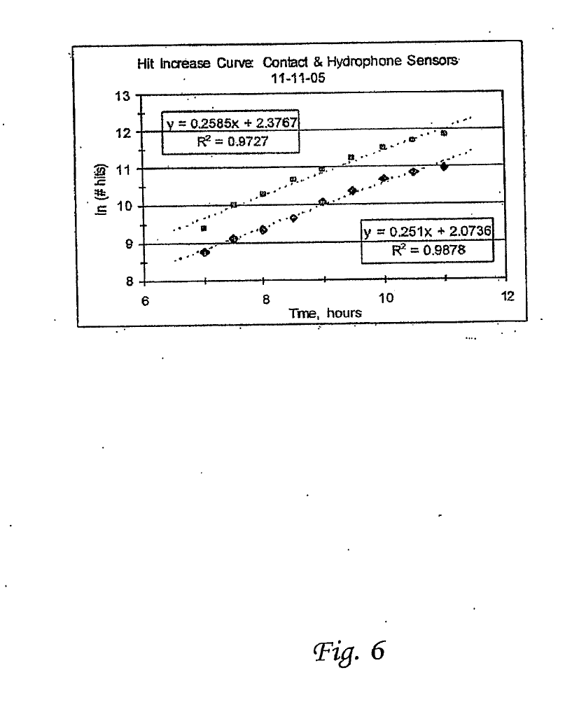

To define whether the increase and corresponding decline in the number of AE hits may be represented by well known exponential increase and decrease functions for cell and microbe growth and death, the natural logarithmic value of the number of hits from both sensors was calculated for the period between 7 and 11 hours and again between 12 and 27 hours. These data are presented in FIGS. 6 & 7.

The data in FIGS. 6 & 7 are logarithmically linear with time, in agreement with known cell and microbe populations in cultures during growth, stationary, and death phases. In addition, the data from the hydrophone and the contact sensor are co-linear, the difference in the slopes during the increasing phase of AE signal detection are less than 3% and difference in the slopes during the decreasing phase of AE signal detection are less than 5%.

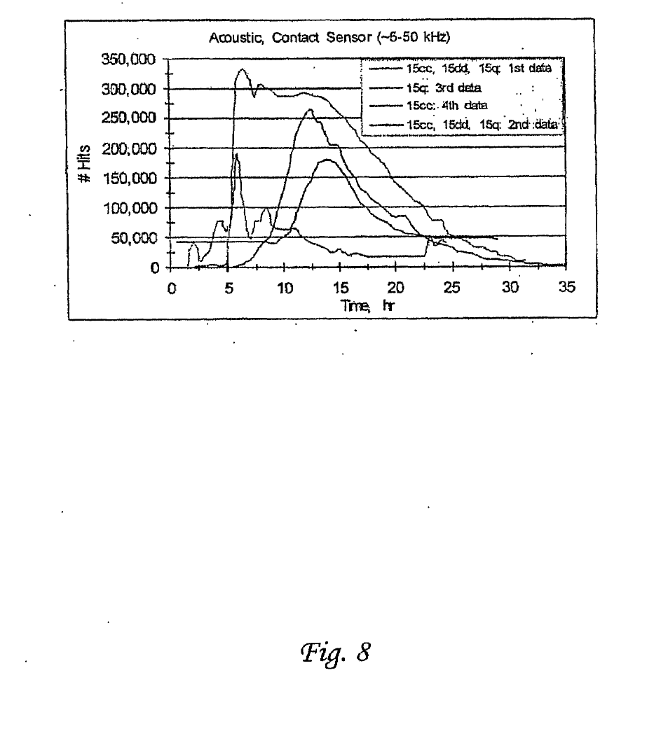

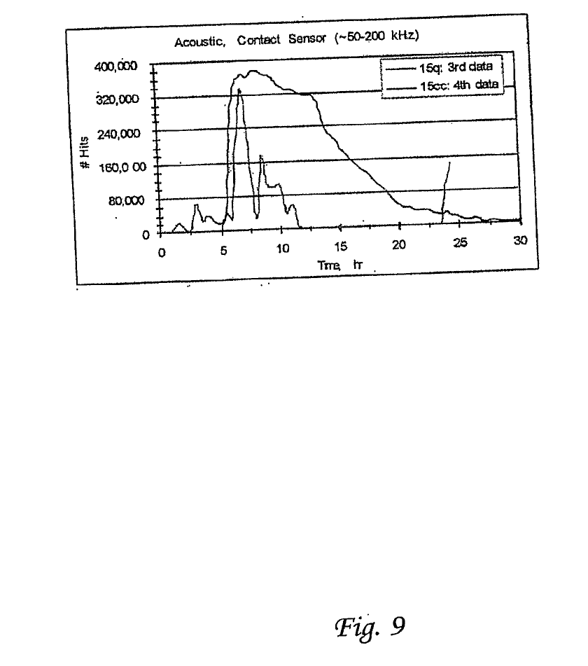

FIGS. 8 and 9 compare the AE #Hits versus time for three different bacteria. The data for E. coli 15Q in both of these figures exhibits the typical cell growth and decline curve that was shown in FIG. 2. The co-mingling of E. coli 15Q, 15CC and 15DD produces a much narrower ‘band’ (i.e. increase and then decrease in the #Hits) and 15CC produces AE having a rapid initial increase in the #Hits. Importantly, the shapes of the two curves for E. coli 15Q, 15CC and 15DD are very similar whereas the shapes of the curves for E. coli 15 Q, 15CC and 15DD are distinct from the shapes of the curves for single cell for E. coli 15Q and 15CC.

The E. coli bacteria were grown in Bacto Tryptic Soy Broth (30 g/l). During AE data acquisition, samples were removed from the growth medium and immediately plated to determine cfu (colony forming units). Samples were serially diluted in phosphate buffer and plated on aerobic 3M petri-film. Counts were determined after the films were incubated at 35° C. for 24 h.

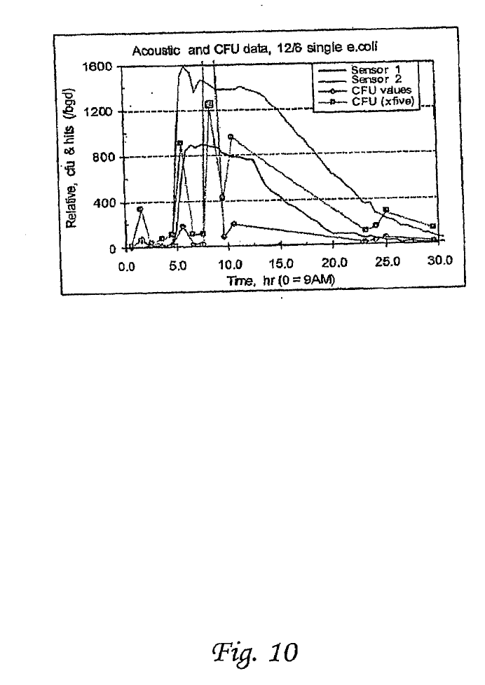

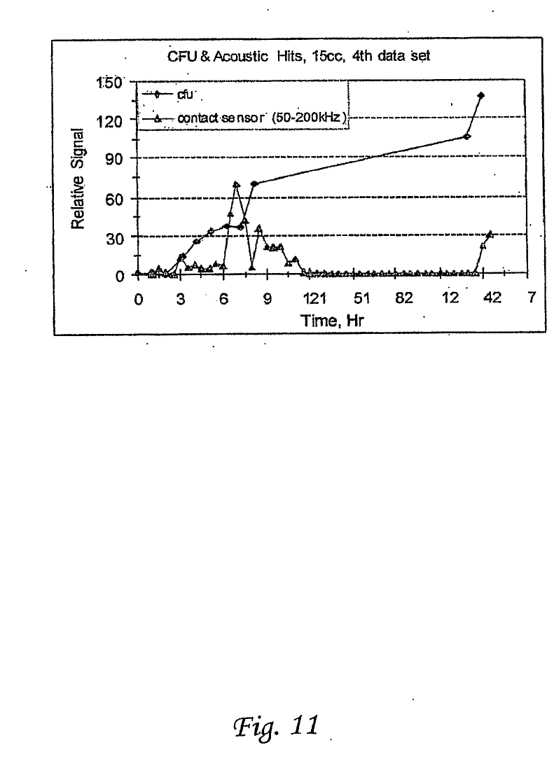

FIG. 10 overlays AE and cfu data for E. coli 15Q; FIG. 11 overlays AE and cfu data for E. coli 15c. The increase and decrease in AE signal coincide with the increase and decrease in cfu's. Additionally, a rapid rise in the cfu's near the end of experiment for E. coli 15CC (at hours ˜24) in FIG. 11 coincided within a 30 fold rise in the AE signal.

Table 3 presents major band intensities in the FFT (fast Fourier transform) spectra of the waveforms for the mixture E. coli 15CC, 15d, 15Q, and for the single cell E. coli 15Q, and E. coli 15CC tests. These characteristic frequencies were identified by visual examination of the FFT spectra after examination of the waveforms to ensure they contained the highest but also representative amplitudes. The data appear to display distinct differences between the three E. coli tests. The mixture 15CC, 15DD, 15Q had high band intensities at 45 and 55 kHz; 15Q had high band intensities at 10, 10, 100, 155, 170 and 220 kHz; 15CC had high band intensities at 40 kHz. Some bands were common in all of the tests even for different E. coli types, e.g. between 23-28 kHz, 21-28 kHz and 68-75 kHz. Other bands were observed in 15Q and 15CC, eg. 80-83 kHz, 90-95 kHz and 145-150 kHz. Even though the low frequency (LF) contact sensor had its highest sensitivity between 5-50 kHz, signals in the 70-95 kHz region could be observed. Similarly, even though the high frequency (HF) contact sensor had its highest sensitivity between ˜50-200 kHz, signals above 200 kHz could be observed.

TABLE 3
Comparison of AE frequencies from different E coli. Some frequencies were common among
all sample periods and for all cultures whereas the values in red were unique to
the cell and microbe colony(s) tested.

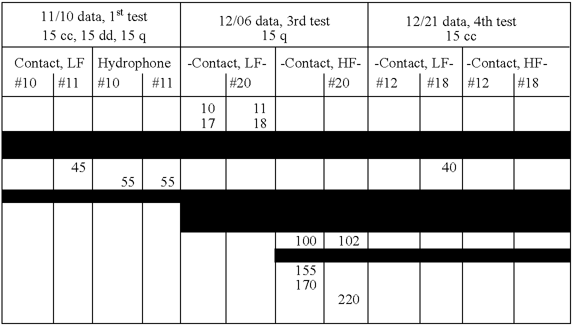

The present method of assessing a biological test specimen may be broadly described as comprising the steps of detecting an AE produced by the biological test specimen and comparing the detected AE to a compilation of AE's produced by known biological sources. For purposes of this document, biological sources may be defined as comprising prokaryotic cells, eukaryotic cells and viruses.

The method may also include the providing of an impulse to the biological test specimen to produce a detectable response. That impulse may be selected from a group of impulses including an electrical impulse, a chemical impulse, a magnetic impulse, an acoustic impulse, a vibratory impulse, an electromagnetic impulse, a mechanical impulse, a radioactive impulse and any combinations thereof. Further, the method includes detecting the AE in a range of between about 20 to about 5,000,000 Hz.

The method includes using a sensor selected from a group of sensors consisting of a piezoelectric sensor, a capacitive sensor, an optoelectronic sensor, a magnetorestrictive sensor and combinations thereof to detect the AE. Multiple sensors may be used to detect the AE. Alternatively, a single sensor may be used by moving the sensor across the biological test specimen.

In addition, the present method includes monitoring various attributes of the test specimen. Those attributes include, but are not limited to:

a. nutrients/chemical and bi-product imbibing and expulsion;

b. cell growth and cell division;

c. cell movement;

d. plasma ejection and imbibing;

e. natural cell wall vibration;

f. cell expiration;

g. quorum sensing communication;

h. transfer of genetic material via sex-pili;

i. growth of pili and transport of biofilm material through the pili;

j. brownium motion;

k. flagella rotation.

The present method is useful for analytical assessments, microbiological containment analysis, clinical diagnoses, and control of processes in which cell and microbe and enzymatic reactions are needed or unwanted for applications like pharmaceutical, food, health care, agriculture, environment and water. Currently, assays of biological systems using “biosensors” require biological specificity-conferring mechanisms within a sensor. This requirement eliminates wide-spread application of a sensor to various reactions or systems. The present method eliminates the specificity requirement on the sensor or substrate and instead relies upon “listening” to biological activity, whether from bacteria, bacteria phages, viruses, animal cells, plant cells, fingi, protozoans and algae and then defining the fingerprint(s) associated with the activity. This enables the subsequent real-time analytical assessment and control of the activity. For example, by defining AE “fingerprints” of a bacterial strain, it is envisioned that rapid assay instrumentation may be created which are strain specific as well as genera specific. These types of assays and data acquisition will allow for the instant recognition of pathogens present in foods and other products, and in clinical settings. AE information that gives estimates or quantitative values for pathogen concentrations and rates of growth enable the prediction of shelf life of products, the effectiveness of sanitation programs within food manufacturing plants, the presence of inappropriate cell and microbe strains and/or activity, the control of processes dependent on specific “healthy” cell and microbe concentrations and the like.

The foregoing description of the preferred embodiments of the present invention have been presented for purposes of illustration and description. It is not intended to be exhaustive or to limit the invention to the precise form disclosed. Obvious modifications or variations are possible in light of the above teachings. The embodiments were chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally and equitably entitled. The drawings and preferred embodiments do not and are not intended to limit the ordinary meaning of the claims in their fair and broad interpretation in any way.

What is claimed:

1. A method of assessing a biological test specimen, comprising:
detecting an acoustic emission produced by said biological test specimen; and
comparing said detected acoustic emission to a compilation of acoustic emissions produced by known biological sources.

2. The method of claim 1, further including providing an impulse to said biological test specimen to produce a detectable response.

3. The method of claim 2, including selecting said impulse from a group of impulses including an electrical impulse, a chemical impulse, a magnetic impulse, an acoustic impulse, a vibratory impulse, an electromagnetic impulse, a mechanical impulse, a radioactive impulse and any combinations thereof.

4. The method of claim 1, including detecting said acoustic emission in a range of between about 20 to about 5,000,000 Hertz.

5. The method of claim 1, including using an acoustic sensor to detect said acoustic emission.

6. The method of claim 1, including using a sensor selected from a group of sensors consisting of a piezoelectric sensor, a capacitive sensor, an optoelectronic sensor, a magnetorestrictive sensor and combinations thereof to detect said acoustic emission.

7. The method of claim 1, including using an optic sensor to detect said acoustic emission.

8. The method of claim 1, including using multiple sensors to detect said acoustic emission.

9. The method of claim 1, including moving a sensor across said biological test specimen in order to detect said acoustic emission.

10. The method of claim 1, including monitoring nutrient/chemical and byproduct imbibing and expulsion of said biological test specimen by detecting and analyzing said acoustic emission.

11. The method of claim 1, including monitoring cell growth and cell division of said biological test specimen by detecting and analyzing said acoustic emission.

12. The method of claim 1, including monitoring cell movement of said biological test specimen by detecting and analyzing said acoustic emission.

13. The method of claim 1, including monitoring plasmid ejection and imbibing of said biological test specimen by detecting and analyzing said acoustic emission.

14. The method of claim 1, including monitoring natural cell wall vibration of said biological test specimen by detecting and analyzing said acoustic emission.

15. The method of claim 1, including monitoring cell expiration of said biological test specimen by detecting and analyzing said acoustic emission.

16. The method of claim 1, including monitoring quorum sensing communication of said biological test specimen by detecting and analyzing said acoustic emission.

17. The method of claim 1, including monitoring transfer of genetic material via sex pili of said biological test specimen by detecting and analyzing said acoustic emission.

18. The method of claim 1, including monitoring growth of pili and transport of biofilm material through said pili of said biological test specimen by detecting and analyzing said acoustic emission.

19. The method of claim 1, including monitoring Browman motion of said biological test specimen by detecting and analyzing said acoustic emission.

20. The method of claim 1, including monitoring flagella rotation of said biological test specimen by detecting and analyzing said acoustic emission.
